(19) United States
(12) Patent Application Publication
(10) Pub. No.: US 2024/0358267 A1
(43) Pub. Date: Oct. 31, 2024

(54) STRUCTURE AND METHOD FOR DETERMINING AND OBTAINING BEST AVAILABLE SIGNAL-TO-NOISE RATIO IN AN ELECTRODERMAL ACTIVITY SENSOR

(71) Applicant: Toyota Motor Engineering & Manufacturing North America, Inc., Plano, TX (US)

(72) Inventors: Hossein Hamidi Shishavan, New Britain, CT (US); Ercan Mehmet Dede, Ann Arbor, MI (US); Frederico Marcolino Quintao Severgnini, Ann Arbor, MI (US); Parveen Singh, Dearborn, MI (US); Paul Donald Schmalenberg, Ann Arbor, MI (US)

(21) Appl. No.: 18/306,489

(22) Filed: Apr. 25, 2023

(51) Int. Cl. A61B 5/0531; A61B 5/00

(52) U.S. Cl. CPC A61B 5/0531; A61B 5/6826; A61B 5/7221; A61B 5/742; A61B 2562/043

(57) ABSTRACT

A wearable device includes an attachment structure, at least one turntable operably connected to the attachment structure, and a pair of insulatively spaced-apart electrodes affixed to the at least one turntable and structured to contact a skin surface of a user. The turntable and electrodes may form parts of an electrodermal activity (EDA) sensor operably connected to the attachment structure. Use of the turntable enables a rotational orientation of the electrodes to be adjusted so as to achieve maximum available area overlap with eccrine glands located in the skin of a user in contact with the electrodes. This maximum available area overlap may provide a best available S/N ratio for an EDA signal generated based on electrode current transmission through the user's skin.

### TECHNICAL FIELD

The subject matter described herein relates to electrodermal activity (EDA) sensors and, more particularly, to the positioning of electrodes in EDA sensors for maximum accuracy of acquired sensor data.

### BACKGROUND

It is known to use electrodermal activity (EDA) sensors to acquire data that may be interpreted to indicate or infer the existence any of a variety of physiological and/or psychological conditions in a user. The EDA sensors may use electrodes in physical contact with the user's skin to transmit an electrical current between the electrodes. Based on an estimate of the resistance or conductance encountered during current transmission, an EDA signal may be generated for analysis of the user's state.

The electrodes may be incorporated into a wearable device, such as a wristband. To help provide the most accurate measurement of electrodermal activity, it is desirable for the generated EDA signal to have a high signal-to-noise (S/N) ratio. It has been found that the S/N ratio of generated EDA signals positively correlates with an orientation of the electrodes in which an area overlap exists between the electrodes and local eccrine glands located under the skin surface beneath the electrodes. However, the electrodes have fixed orientations and positions on the user's skin while the device is being worn. Thus, depending on the random relationships between the positions and orientations of the electrodes and the locations of the eccrine glands in the skin surface below the electrodes, there may be insufficient area overlap of the electrodes with the eccrine glands to provide an S/N ratio sufficient for accurate EDA readings.

### SUMMARY

In one aspect of the embodiments described herein, a wearable device is provided. The wearable device includes an attachment structure, at least one turntable operably connected to the attachment structure, and a pair of insulatively spaced-apart electrodes affixed to the at least one turntable and structured to contact a skin surface of a user.

In another aspect of the embodiments described herein, an electrodermal activity (EDA) sensor is provided. The sensor includes at least one turntable and a pair of insulatively spaced-apart electrodes affixed to the at least one turntable and structured to contact a skin surface of a user.

In yet another aspect of the embodiments described herein, a method is provided for determining electrodermal activity (EDA) associated with a user of a wearable device incorporating a pair of electrodes rotatably mounted to an attachment structure of the wearable device. The method includes a step of determining a plurality of values of S/N ratio, with each value of S/N ratio being determined from an associated EDA signal generated when the electrodes are in an associated rotational orientation. Each value the plurality of values of S/N ratio is compared with all of the other values of the plurality of values of S/N ratio to determine a highest S/N ratio of the plurality of values of S/N ratio. An instruction is then generated to rotate the electrodes so as to orient the electrodes in the rotational orientation associated with the highest S/N ratio of the plurality of values of S/N ratio, so that the electrodes generate in EDA signals while oriented in the rotational orientation associated with the highest S/N ratio during operation of the wearable device.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

It will be appreciated that for simplicity and clarity of illustration, where appropriate, reference numerals may have been repeated among the different figures to indicate corresponding or analogous elements. Also, similar reference numerals appearing in different views may refer to similar elements appearing in those views. In addition, the discussion outlines numerous specific details to provide a thorough understanding of the embodiments described herein. Those of skill in the art, however, will understand that the embodiments described herein may be practiced using various combinations of these elements.

FIG. 1 is a block schematic diagram of an example wearable device in accordance with embodiments described herein.

FIG. 2 is a schematic perspective view of a wearable device in the form of a finger ring including an electrode turntable mounted in an attachment structure.

FIG. 3 is a partial schematic cross-sectional view of the turntable mounted in the finger ring of FIG. 2.

FIG. 4A is a schematic plan view showing turntable-mounted electrodes and a turntable in a first rotational orientation and overlaid and in contact with a skin surface of a user, and also showing representations of eccrine glands of the skin surface beneath the electrodes.

FIG. 4B is the schematic plan view of FIG. 4A, showing the electrodes rotated to a second rotational orientation by rotating the turntable to a second rotational orientation.

FIG. 5A is a schematic partial plan view of an inner surface of the attachment structure of the finger ring of FIG. 2, showing the turntable at a reference rotational orientation of 0°.

FIG. 5B is the schematic plan view of FIG. 5A illustrating progressive rotation of the turntable to rotational orientations of 45°, 90°, and 135° relative to the reference rotational orientation shown in FIG. 5A.

FIG. 6 is a flow diagram illustrating a procedure for collecting signal-to-noise (S/N) ratios at a plurality of associated rotational orientations of the turntable in the wearable device embodiment shown in FIGS. 1-5B.

FIG. 7 is a flow diagram illustrating a method of orienting the turntable in the embodiment shown in FIGS. 1-5B to achieve the highest S/N ratio out of a plurality of S/N ratios.

FIG. 8A is a partial schematic plan view of a portion of an inner surface of a finger ring showing features of an alternative embodiment of the wearable device.

FIG. 8B is a partial schematic cross-sectional view of the portion of the finger ring shown in FIG. 8A.

FIG. 9 is a flow diagram illustrating a procedure for collecting signal-to-noise (S/N) ratios at a plurality of associated rotational orientations of the turntables in the wearable device embodiment shown in FIGS. 8A and 8B.

FIGS. 10A-10D are schematic plan views of the embodiment of FIGS. 8A and 8B, illustrating progressive rotation of a second turntable to rotational orientations of 45°, 90°, and 135° relative to the 0° reference rotational orientation shown in FIG. 8A, while the first turntable is held at 0°.

FIGS. 10E-10H are schematic plan views of the embodiment of FIGS. 8A and 8B, illustrating progressive rotation of the second turntable to rotational orientations of 45°, 90°, and 135° relative to the 0° reference rotational orientation after rotation of the first turntable to 45°.

FIG. 11 is a flow diagram illustrating a method of determining electrodermal activity (EDA) associated with a user of a wearable device incorporating a pair of electrodes rotatably mounted to an attachment structure of the wearable device.

### DETAILED DESCRIPTION

Described herein are embodiments of a wearable device including an attachment structure, at least one turntable operably connected to the attachment structure, and a pair of insulatively spaced-apart electrodes affixed to the at least one turntable and structured to contact a skin surface of a user. The turntable(s) and electrodes may form parts of an electrodermal activity (EDA) sensor operably connected to the attachment structure. Use of the turntable(s) enables a rotational orientation of the electrodes to be adjusted so as to achieve maximum available area overlap with eccrine glands located in the skin of a user in contact with the electrodes. This maximum available area overlap may provide a best available S/N ratio for an EDA signal generated based on electrode current transmission through the user's skin.

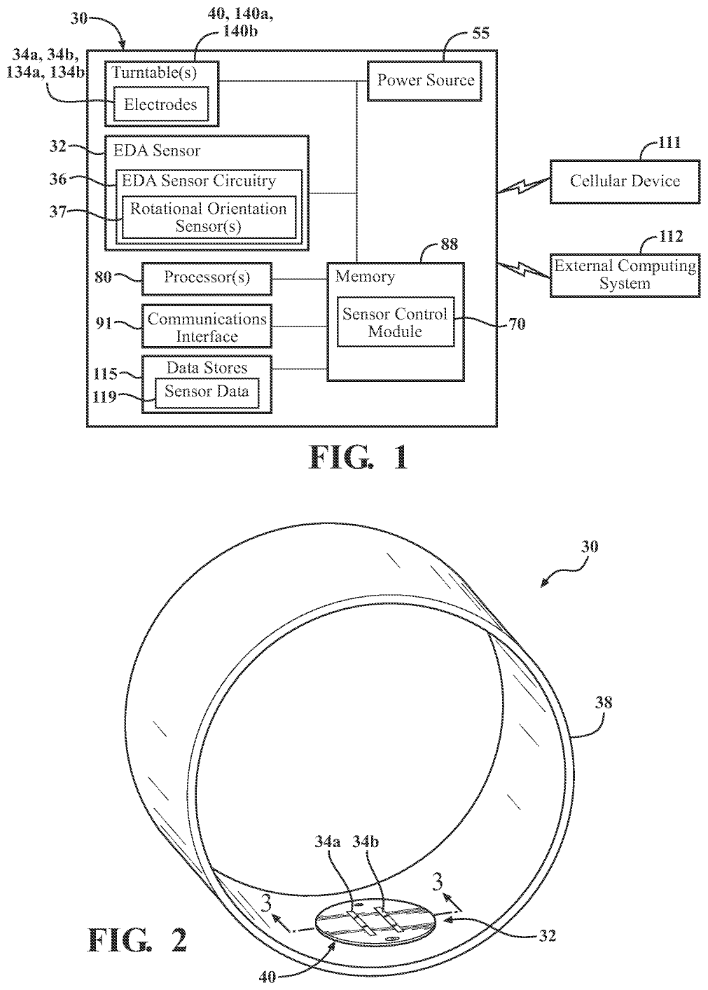

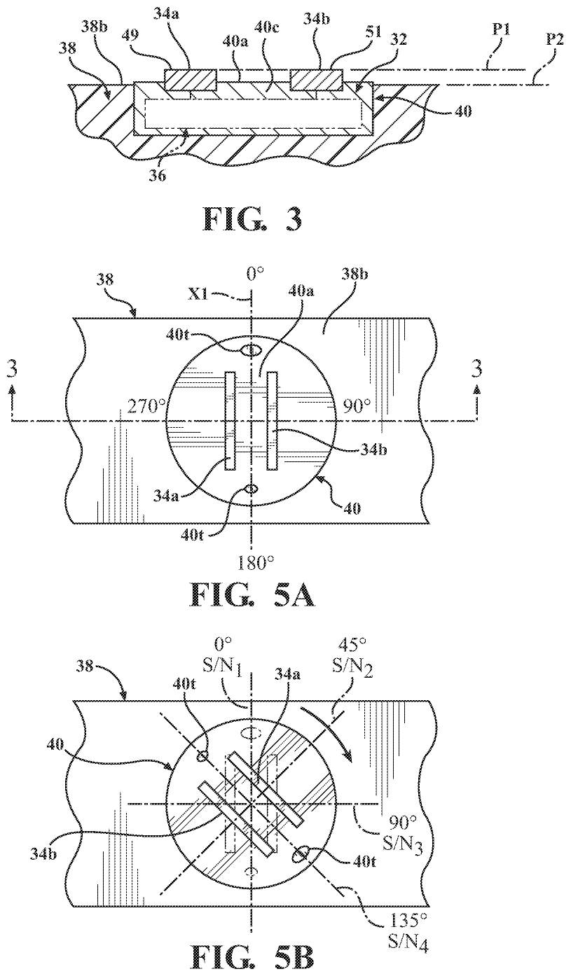

FIGS. 1 and 2 illustrate an example of a wearable device 30 in accordance with embodiments described herein. In embodiments described herein, the wearable device 30 may include an attachment structure 38. The wearable device may also include at least one turntable operably connected to the attachment structure. In the embodiment shown in FIG. 2, the wearable device 30 includes a single turntable 40 operably connected to the attachment structure. In addition, a pair of insulatively spaced-apart electrodes 34a. 34b may be affixed to the turntable 40 and structured to contact a skin surface of a user. The turntable 40 and electrodes 34a, 34b may form parts of an electrodermal activity (EDA) sensor 32 operably connected (or incorporated into) to the attachment structure. The EDA sensor 32 and associated EDA sensor circuitry 36 (shown in FIG. 3) may be structured to provide an EDA signal responsive to physical contact between the electrodes 34a, 34b and a skin surface of a user.

The EDA sensor 32 may be operably connected to the attachment structure 38 so that the EDA electrodes 34a, 34b are positioned and maintained in contact with the user's skin when the attachment structure 38 is worn by the user. Referring to FIG. 2, in one or more particular arrangements, the attachment structure 38 is in the form of a finger ring (i.e., a ring worn by a user on the user's finger). In other particular arrangements, the attachment structure may be in the form of a wrist band or other structure.

As used herein, “sensor” means any device, component and/or system that can detect, and/or sense something. The EDA sensor 32 described herein can be configured to detect, and/or sense in real-time. As used herein, the term “real-time” means a level of processing responsiveness that a user or system senses as sufficiently immediate for a particular process or determination to be made, or that enables a processor(s) to keep up with some external process.

For purposes described herein, a “turntable” is a generally cylindrical platform or base rotatably mounted to the attachment structure 38. For purposes described herein “rotatable mounting” of the turntable 40 to the attachment structure 38 means that the turntable is structured to be rotatable with respect to the attachment structure 38 and remains affixed to the attachment structure during turntable rotation and use of the EDA sensor 32 for the purposes described herein. For example, the turntable 40 may include a screw-type mounting structured for mating engagement with a complementarily-threaded cavity formed in the attachment structure 38. In other arrangements, the turntable 40 may be positioned in a complementary receptacle 38a formed in the attachment structure 38 and secured in the receptacle so as to be rotatable by a user, but not removable from the receptacle. The turntable 40 may then be rotated using a suitable tool, such as a screwdriver or a dedicated rotation tool specially designed for the manipulating the turntable. For example, portions of a tool may be inserted into complementary tool holes 40t (FIG. 5A) to rotate the turntable. Use of the turntable 40 enables rotation of the electrodes 34a, 34b with respect to the user's skin, as described herein.

The turntable 40 may be mounted along an inner surface 38b of the attachment structure (i.e., a surface designed to face toward and/or contact a skin surface of a user when the wearable device 30 is being worn). The turntable 40 may have an inner, exposed face 40a structured to face toward and contact a skin surface of a user when the wearable device 30 is being worn.

Portions of the turntable in contact with the electrodes 34a, 34b may be formed from one or more materials that can electrically insulate the electrodes. Any suitable electrically insulating material having high resistivity such as air or space (e.g., a vacuum), nonconductive materials (e.g., plastics, nylons, ceramics, etc.), or semiconducting materials in relatively nonconductive states can be used. Other portions of the turntable may be formed form the same material or from a different material or materials suitable for the turntable functions described herein.

As stated previously, a pair of insulatively spaced-apart electrodes 34a, 34b may be affixed to the turntable 40 and structured to contact a skin surface of a user. As used herein, the term “insulatively spaced-apart” means that the electrodes 34a, 34b are mounted in the turntable 40 so as to be physically spaced apart by an electrical insulator 40c, such as an insulating ceramic or polymer material, for example. In one or more arrangements, the electrodes 34a, 34b may be rectangular in shape and mounted on the turntable 40 arranged so as to extend parallel to each other.

In one or more arrangements, the electrodes 34a. 34b may be formed from gold, a gold alloy, silver, a silver alloy, or any other suitable material(s). However, the electrodes 34a, 34b may include one or more conductive polymers, semiconductors, or other conductive materials to minimize electrical resistance and/or electrical polarization. It will be appreciated that any suitable conductive material or materials may be utilized. FIG. 3 is a partial cross-sectional view of the turntable 40 mounted in the attachment structure 38. Referring to FIG. 3, the electrodes 34a, 34b may be mounted to the turntable 40 so as to extend along (or from) the turntable inner face 40a. When the electrodes 34a, 34b are mounted on the turntable 40, a first electrode 34a of the pair of electrodes may have a first inwardly-facing surface 49 structured to contact a skin surface of a user when the user is wearing the wearable device 30. Similarly, a second electrode 34b of the pair of electrodes may have a second inwardly-facing surface 51 structured to contact a skin surface of the user when the user is wearing the wearable device. In particular arrangements, the first inwardly-facing surface 49 and the second inwardly-facing surface 51 are structured to be coplanar along a flat plane P1. This arrangement helps ensure that both of electrodes 34a, 34b simultaneously contact a skin surface of the user when the user is wearing the wearable device 30.

In more particular arrangements, a plane P2 defined by the portion of the turntable inner face 40a separating the first electrode 34a from the second electrode 34b may be spaced apart from, and parallel to, plane P1 including the inwardly-facing surfaces 49 and 51 of the first electrode 34a and the second electrode 34b. This arrangement may permit the electrodes 34a, 34b to extend slightly deeper into the skin surface for enhanced contact between the electrodes and the skin surface when the user is wearing the wearable device 30. In particular arrangements, the spacing between the planes P1 and P2 may be in the range 0-0.020 inches.

The size of an electrode can determine the effective electrode-skin contact area, the signal-to-noise ratio of the generated EDA signal, and the sensitivity of the EDA sensor. To enhance these properties, it is generally beneficial to have relatively larger electrodes. However, the sizes of the electrodes 34a, 34b may be constrained by the surface area available on the turntable inner face 40. The electrodes should not be too small because higher current densities resulting from small electrode-skin contact areas may increase signal generation errors due to factors such as counter emf. In one or more arrangements described herein, and for a wearable device in the form of a finger ring, two electrodes may be structured so as to have inwardly-facing surfaces of equal areas, with the sum of the areas of the inwardly-facing surfaces being equal to about 0.15 centimeter².

Regarding the spacing between the electrodes, increasing the electrode spacing distance may reduce standard deviations between measurements, thereby providing more repeatable results. However, a relatively smaller inter-electrode space leads to a stronger electric field within a smaller skin volume. These competing effects may be balanced according to the requirements of a given application, and in consideration of the surface area available on the turntable inner surface.

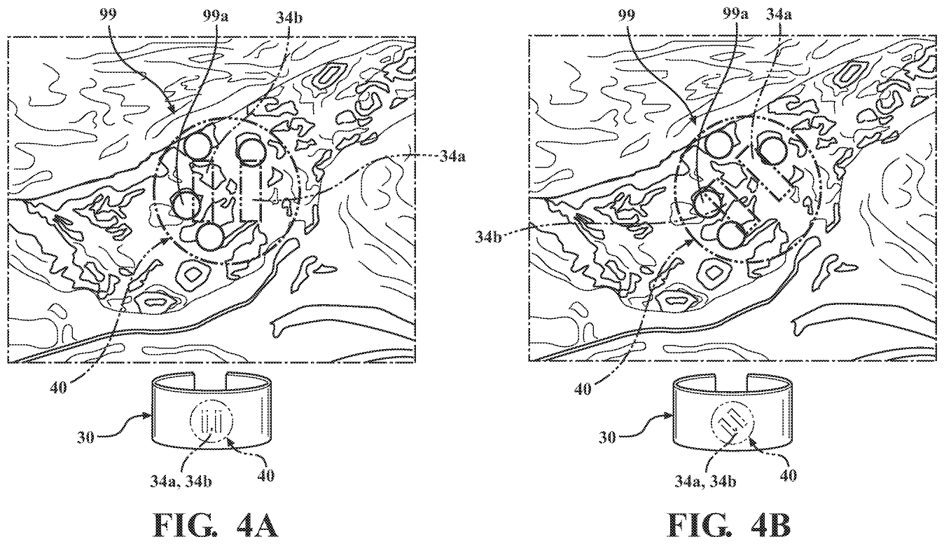

To help provide the most accurate measurement of electrodermal activity, it is desirable for the generated EDA signal to have a high signal-to-noise (S/N) ratio. Referring to FIGS. 4A and 4B, it has been found that the S/N ratio of generated EDA signals positively correlates with an orientation of the electrodes in which a maximum area overlap exists between the electrodes and local eccrine glands located beneath the electrodes (i.e., where as much of the total electrode area as possible overlaps the eccrine glands located beneath the skin surface in contact with the electrodes).

In arrangements described herein, to adjust the area overlap between the electrodes 34a, 34b and the eccrine glands, the rotational orientation of the electrodes may be adjusted by rotating the turntable 40. For example, FIG. 4A is a schematic plan view showing turntable-mounted electrodes 34a, 34b overlaid and in contact with a skin surface 99. FIG. 4A also shows representations of eccrine glands 99a located under the skin surface beneath the electrodes. In FIG. 4A, the electrodes 34a, 34b are in a first orientation (associated with a first rotational orientation of the turntable 40) in which an area overlap between the electrodes 34a, 34b and local eccrine glands 99a has a first value. An EDA signal generated from the electrodes 34a, 34b in this first orientation may have first S/N ratio. FIG. 4B shows the same electrodes 34a, 34b rotated to a second rotational orientation (by rotating the turntable 40 to a second rotational orientation). In the second orientation, a greater amount of the total electrode area overlaps the local eccrine glands 99a. In such a case, it has been found that the S/N ratio of an EDA signal generated from the electrodes 34a, 34b in this second orientation will be higher than the first S/N ratio.

Thus, to find the highest available S/N ratio for a selected wearing position of the finger ring, the turntable 40 may be rotated to several sample rotational orientations to re-orient the electrodes 34a, 34b with respect to the local eccrine glands 99a. At each sample rotational orientation, an S/N ratio may be determined based on an EDA signal generated while in the sample rotational orientation. These S/N ratios may be stored in a memory in association with the respective rotational orientations of the turntable 40. The stored S/N ratios may then be compared with each other to determine a highest S/N ratio of the stored S/N ratios. The rotational orientation corresponding to this highest S/N ratio may be taken as an optimum operating rotational orientation for this particular finger of this particular user. The turntable 40 may then be rotated to the rotational orientation corresponding to the highest S/N ratio and may remain set to this rotational orientation during wearing of the wearable device 30 by this user on the same finger. Due to differences in local eccrine gland locations and configurations, the procedure just described would need to be repeated if the same user chose to wear the ring on a different finger or if the ring was to be worn by a different user.

Referring again to FIG. 3, in one or more arrangements, the wearable device 30 may include EDA sensor circuitry 36 communicatively coupled to the electrodes 34a, 34b and configured to generate an EDA signal using information acquired through physical contact between the electrodes 34a, 34b and a skin surface of a user. An EDA signal may be a signal indicative of electrodermal activity associated with the user wearing the wearable device 30. For example, the EDA signal may be indicative of a measurement of conductance or resistance associated with one or more dermal layers of the user's skin. The user's perspiration can provide electrical contact between the electrodes 34a. 34b and the skin surface of the user, thereby facilitating ionic flow between the electrodes and the skin surface. The EDA sensor circuitry 36 and electrodes 34a. 34b can induce a current through one or more dermal layers of a user's skin. The current transmitted between the electrodes 34a, 34b through the user's skin can be measured and correlated in a known manner to determine conductance, resistance, or another measure indicative of sympathetic nervous system activity.

EDA sensor circuitry 36 can include various components such as amplifiers, filters, charging circuits, sense nodes, and other elements configured to sense one or more electrical characteristics of a user responsive to current transmission through the electrodes. EDA sensor circuitry 36 can be implemented as voltage sensing circuitry, current sensing circuitry, capacitive sensing circuitry, resistive sensing circuitry, etc. In one or more arrangements, at least a portion of the EDA sensor circuitry 36 may be incorporated into the turntable structure 40 as shown in FIG. 3. In one or more arrangements, one or more elements of the EDA sensor circuitry 36 may be incorporated into the attachment structure 38 of the wearable device 30, exterior of the turntable structure 40. In one or more arrangements, the EDA sensor circuitry 36 may cause EDA signals to be generated automatically and periodically whenever the electrodes 34a, 34b are in physical contact with a user's skin surface. In some particular arrangements, the sensor control module 70 (described in greater detail below) may cause EDA signals to be generated at specific times or interact with the EDA sensor circuitry 36 to cause generation of one or more EDA signals.

In one or more arrangements, the EDA sensor circuitry 36 may include a rotational orientation sensor 37 configured to detect or measure a rotational orientation of the turntable 40 relative to a reference rotational orientation of the turntable. The reference rotational orientation of the turntable 40 may be stored in memory so that a final calibrated orientation of the turntable (with respect to the reference rotational orientation) for a given user and finger may be saved after calibration of the wearable device 30 as described herein. Assigning unique numerical values to each rotational orientation of the turntable 40 enables an S/N ratio to be associated with each rotational orientation.

Each rotational orientation of the turntable 40 may have a unique value, from 0° to 359°, based on the reference orientation. In the example shown in FIG. 5A, a reference rotational orientation value of 0° is assigned to an arrangement where an axis X1 bisecting a perpendicular distance between the electrodes 34a, 34b extends perpendicular to an edge of the attachment structure 38. However, the 0° reference orientation value can be assigned to any desired rotational orientation.

In one or more arrangements, the rotational orientation sensor 37 may employ a digital potentiometer or other suitable circuit arrangement to determine the rotational orientation of the turntable. The rotational orientation sensor 37 (or a processor(s) communicably coupled to the sensor) may be configured to determine an amount of turntable rotation (i.e., from a first rotational orientation to a second, different rotational orientation) by determining an angular difference between the first and second successive rotational orientations.

Referring again to FIG. 1, in one or more arrangements, the wearable device may also include a power source 55 (such as a battery) operably connected to other elements of the wearable device 30 and configured for powering the various operations performed by the processor(s) 80, the EDA sensor circuitry 36, and other active elements of the wearable device 30. For example, power source 55 may be coupled to EDA sensor circuitry 36 to provide power to sensing circuitry to enable transmission of an electric current through electrodes 34a, 34b. The power source 55 may be removable or embedded within the wearable device 30.

Referring to FIG. 1, the wearable device 30 can include one or more processor(s) 80. The wearable device 30 can also include one or more data stores 115 for storing one or more types of data. The data store(s) 115 can include volatile and/or non-volatile memory. Examples of suitable data store(s) 115 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory) or any other suitable storage medium, or any combination thereof. The data store(s) 115 can be a component of the processor(s) 80, or the data store(s) 115 can be operably connected to the processor(s) 80 for use thereby. The term “operably connected,” as used throughout this description, can include direct or indirect connections, including connections without direct physical contact. The one or more data store(s) 115 can include sensor data 119. In this context, “sensor data” means any information about any sensors that the wearable device 30 is equipped with, including the capabilities and other information about such sensors.

The wearable device 30 can include a sensor control module 70. The sensor control module 70 can be implemented as computer-readable program code that, when executed by processor(s) 80, implement one or more of the various steps and/or processes described herein. The module can be a component of the processor(s) 80, or the module can be in a memory 88 operably connected the processor(s) 80. The module can include instructions (e.g., program logic) executable by one or more processor(s) 80. Alternatively, or in addition, one or more of data store(s) 115 may contain such instructions.

Generally, a module, as used herein, includes routines, programs, objects, components, data structures, and so on that perform particular tasks or implement particular data types. In further aspects, a memory generally stores the noted modules. The memory associated with a module may be a buffer or cache embedded within a processor(s), a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions. The processor(s) 80, the memory 88 and the sensor control module 70 can be operably connected to communicate with the other elements of the wearable device.

In one or more arrangements, the sensor control module 70 may be configured to (i.e., the module may include computer-readable instructions that when executed by the processor(s) 80 cause the processor(s) to), using an EDA signal generated while the turntable 40 is in a current rotational orientation, determine an S/N ratio associated with the current rotational orientation. The sensor control module 70 may also be configured to, determine if a need exists to rotate the turntable 40 a predetermined amount from a current rotational orientation. The sensor control module 70 may also be configured to, if a need exists to rotate the turntable 40 a predetermined amount from a current rotational orientation, generate an instruction to rotate the turntable 40 the predetermined amount. The sensor control module 70 may also be configured to determine when the turntable 40 has been rotated the predetermined amount. The sensor control module 70 may also be configured to, responsive to a determination that the turntable 40 has been rotated the predetermined amount, generate an instruction to the user to stop rotating the turntable 40.

The sensor control module 70 may also be configured control (or prompt operation of) a display device so as to display an instruction to rotate the turntable 40 on the display device. The sensor control module 70 may also be configured to determine a plurality of values of S/N ratio based on successive rotations of the turntable 40 to different rotational orientations, with each value of S/N ratio being associated with a different rotational orientation of the turntable 40. The sensor control module 70 may also be configured to compare each value of the plurality of values of S/N ratio with all of the other values of the plurality of values of S/N ratio to determine a highest S/N ratio of the plurality of values of S/N ratio. The sensor control module 70 may also be configured to generate an instruction to rotate the turntable 40 so as to orient the turntable in a rotational orientation associated with the highest S/N ratio of the plurality of values of S/N ratio.

The communications interface 91 may be configured to enable and/or facilitate communication between the components of the wearable device 30 and entities (such as cloud facilities, cellular and other mobile communications devices, external computing systems, etc.) exterior of the wearable device. For example, the communications interface 91 may enable interaction between a wearer of the wearable device and a cellular device 111 functioning as a screen display to illustrate calibration procedures for the wearable device 30. The communications interface 91 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. The communications interface 91 can be a wired and/or wireless network interface.

Referring again to FIG. 1, in one or more arrangements, the sensor control module 70 may be configured to interface with a cellular device 111 configured to perform one or more of the functions described herein. For example, in some arrangements, the sensor control module 70 may be configured to operate in cooperation with a suitable application downloaded onto the cellular device 111 to process EDA signals generated by the EDA sensor circuitry 36 to determine associated S/N ratios of the signals. In this respect, the wearable device 30 may utilize the computing power available in the cellular device 111. In other aspects, an external computing system 112 may perform one or more of the functions just described regarding the cellular device 111. In some arrangements, the cellular device 111 may be configured to operate in cooperation with the sensor control module 70 to function as a display device for displaying (to a user) instructions and/or other information relating to operation of the wearable device 30.

The cellular device 111 and/or the computing system 112 may each also be configured to perform additional functions not mentioned above. The cellular device 111 and the computing system 112 may each communicate with the wearable device 30 via the communications interface 91. For example, wearable device 30 may transmit data indicative of a user's electrodermal activity to one or more external devices (such as cellular device 111 and/or external computing system 112) in example embodiments. When electrodermal activity is detected by EDA sensor circuitry 36 of the wearable device 30, data representative of the electrodermal activity may be communicated, via the communications interface 91 and a suitable communications network, to an external device. The external device may then analyze the data to determine information associated with a user's electrodermal activity. The data and/or one or more control signals may then be utilized to cause the external device to initiate a particular functionality. Generally, the communications interface 91 may be configured to communicate data, such as EDA data, over wired, wireless, or optical networks to and from external devices.

Since the electrodes 34a. 34b are affixed to the turntable 40 for rotation in correspondence therewith, each 180° rotation of the turntable 40 from any given starting orientation of the turntable will provide the same orientation of the electrodes 34a. 34b (for purposes of overlapping the local eccrine glands under the skin below the electrodes). Thus, a value of the S/N ratio determined at 180° will be the same as a value of S/N ratio determined at 0°. Also, each rotational orientation of the electrodes 34a, 34b may be associated with two different rotational turntable orientations spaced apart 180°. For example, the rotational orientation of the electrodes shown in FIG. 5A may be associated with rotational orientations of the turntable at 0° and 180°. In addition, since each rotational orientation of the turntable 40 has a unique value, rotating the turntable to rotational orientations of up to 179 ° in either direction (clockwise or counterclockwise) from any given starting orientation (i.e., taken to be at a 0° starting orientation) will enable determination of a unique value of S/N ratio associated with each rotational orientation of the turntable 40 between 0° and 179°.

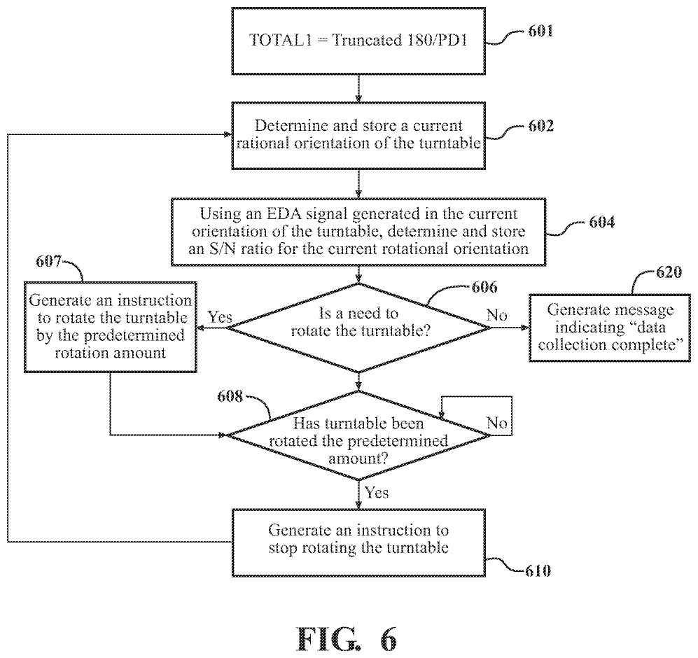

FIGS. 5A, 5B, and 6, in combination, illustrate a method of acquiring a plurality of S/N ratios at various rotational orientations of the turntable 40 for evaluation. In this scenario, the turntable 40 is to be rotated a predetermined amount equal to 45° between S/N ratio readings. However, other predetermined angular amounts may be used. Also, in the example shown, the turntable 40 is shown starting in the 0° orientation, although the turntable may start the calibration process in any random orientation.

To begin, in block 601 (FIG. 6), a user may start a calibration routine for the wearable device, for example by interfacing with an external computing device or by operating a switch (not shown) incorporated into the wearable device. Starting of the calibration routine may initialize a variable TOTAL1 by taking the quotient 180/PD1 (where PD1=the predetermined rotation amount, in degrees) and truncating any fractional portion to obtain an integer value of the quotient. For example, for PD1=35°, 180/PD1=5.14 and TOTAL1=5 (the truncated value of the quotient). For PD1=45° as shown in the example, 180/PD1=4. The variable TOTAL1 becomes the total number of S/N readings that should be taken for calibration purposes. In addition, a counter CC1 may be initialized to zero.

Referring to FIG. 6, in a next step (block 602), the sensor control module 70 may determine and store a current rotational orientation of the turntable 40. Thus, per the example shown in FIG. 5A, the 0° orientation may be stored as the current orientation. In a next step (block 604), the sensor control module 70 may, using an EDA signal generated in the current rotational orientation of the turntable 40, determine and store an S/N ratio for the current rotational orientation. This S/N ratio may be stored in a memory 88 in operative association with the current turntable rotational orientation. In this manner, each S/N ratio may be associated with a given rotational orientation of the turntable 40. In the example shown, a ratio S/N1 may be stored in association with the 0° turntable orientation.

Next (block 606), the sensor control module 70 may determine if there is a need to rotate the turntable 40 to generate additional sample values of S/N ratio. To this end, the sensor control module 70 may increment a counter CC1 by 1 (indicating that an S/N ratio has just been determined) and compare the value of CC1 to the value of TOTAL1. If CC1=TOTAL1, then no additional S/N readings need to be taken for calibration. Then, the sensor control module 70 may (in block 620) generate message indicating all of the needed S/N ration values have been obtained (i.e., that data collection is complete). However, if CC1<TOTAL1, then one or more additional S/N readings should be taken for calibration. The sensor control module 70 may then (in block 607) generate an instruction to rotate the turntable by the predetermined rotation amount.

In particular arrangements, the sensor control module 70 may be configured to control a display device (such as a cellular device 111 in operative communication with the sensor control module) or to prompt operation of a display device so as to display the turntable rotation instruction(s) on the display device. The instruction may include elements such as how to use a turntable rotation tool (not shown) provided with the wearable device 30 and other instructions which may be conveyed clearly in an audio/visual format.

Following generation of the instruction in block 607, the user may proceed to rotate the turntable 40 responsive to the instruction. The sensor control module 70 may be configured to constantly monitor (in block 608) the rotational orientation of the turntable 40 (in cooperation with the rotational orientation sensor 37, for example) to determine when the turntable 40 has been rotated the predetermined amount (in this case, by) 45°. When the turntable 40 has been rotated the predetermined amount, the sensor control module 70 may (in block 610) generate an instruction to stop rotating the turntable 40. Control may then proceed back to block 602 to repeat the steps just described, until CC1=TOTAL1.

Referring to FIG. 5B, the process just described may be performed for additional turntable orientations of 45°. 90°, and 135°. When the turntable reaches an angle of 135° and an associated S/N ratio has been determined, CC1 will have been incremented to 4, and S/N ratio values of S/N1 at 0°, S/N2 at 45°, S/N3 at 90°, and S/N4 at 135° will have been recorded. When CC1=TOTAL1=4, the turntable 40 does not need to be rotated any further to provide additional S/N ratio values. The sensor control module 70 can now proceed to determining the highest S/N ratio value out of all the values just collected.

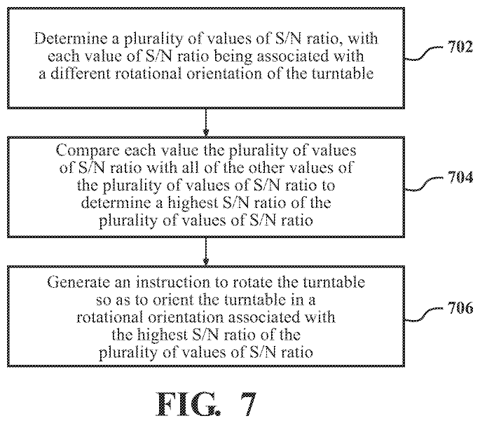

FIG. 7 is a flow diagram illustrating a method of orienting the turntable 40 to achieve the highest S/N ratio out of a plurality of S/N ratios. In one or more arrangements, the sensor control module 70 and the processor(s) 80 may be configured to perform the method described in FIG. 7. In one or more arrangements, the plurality of S/N ratios may be ratios previously acquired and stored during performance of the sequence described in FIG. 6. However, the method may be performed using any group of S/N values.

Referring to FIG. 7, in a first step (block 702), a plurality of values of S/N ratio may be determined (for example, using the method described with regard to FIG. 6), with each value of S/N ratio being associated with a different rotational orientation of the turntable 40. Next (in block 704), the sensor control module 70 may compare each value the plurality of values of S/N ratio with all of the other values of the plurality of values of S/N ratio to determine a highest S/N ratio of the plurality of values of S/N ratio. In a next step (block 706), the sensor control module 70 may generate an instruction to rotate the turntable 40 so as to orient the turntable in a rotational orientation associated with the highest S/N ratio of the plurality of values of S/N ratio.

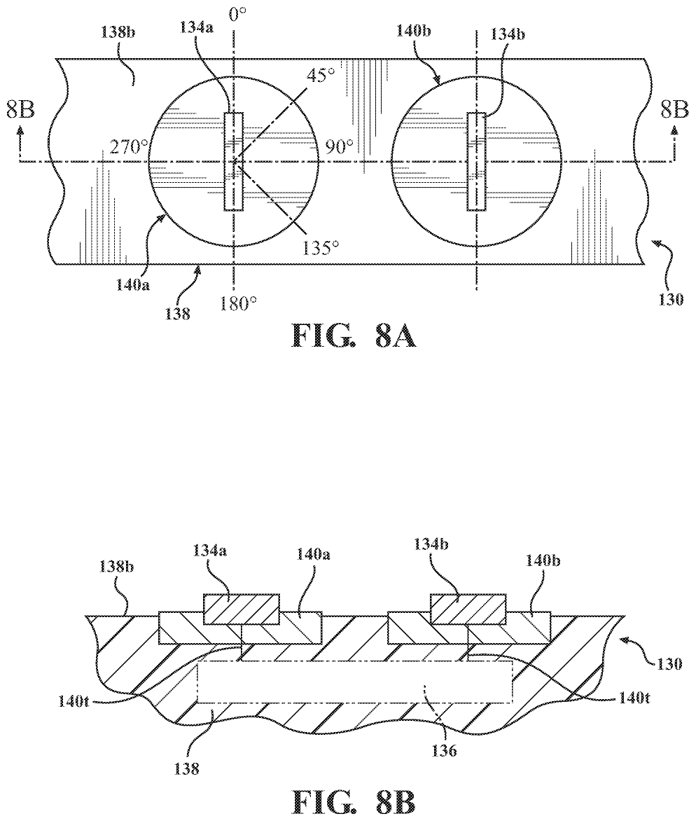

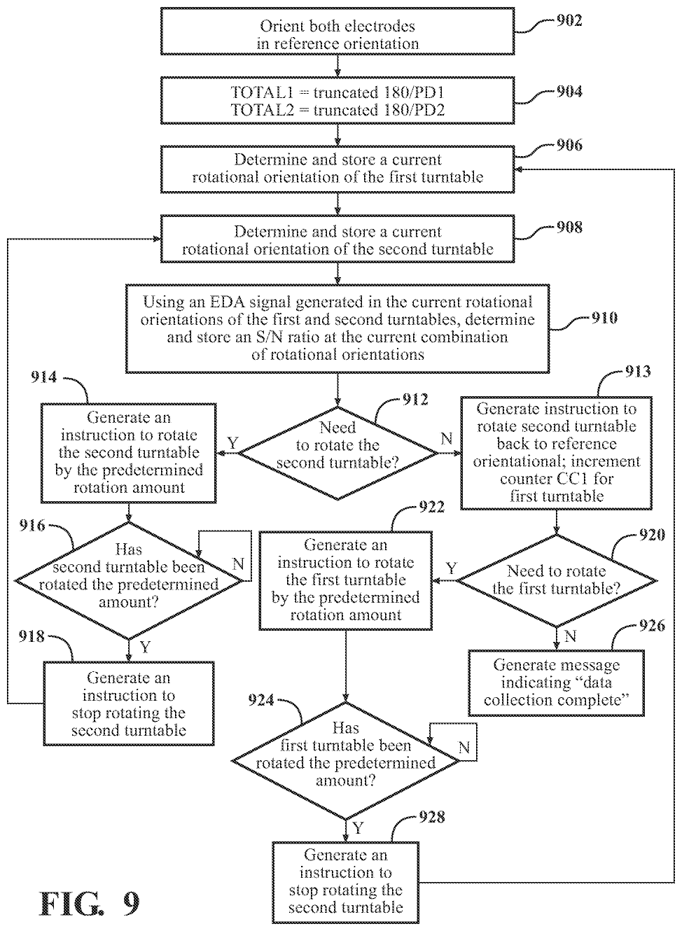

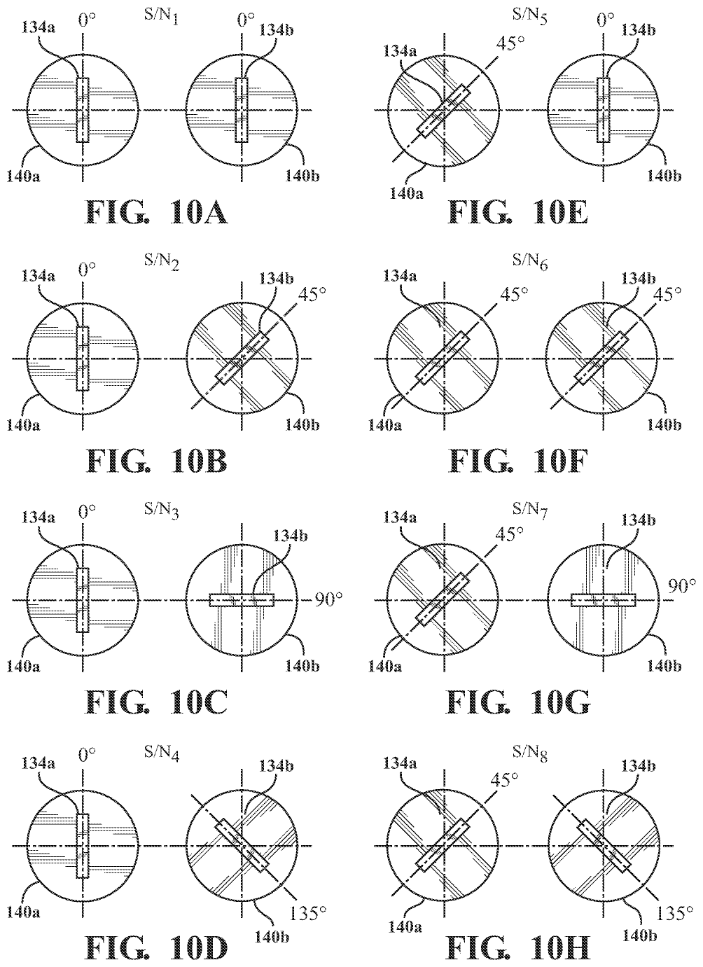

FIG. 8A is a schematic plan view of an inner surface of a finger ring attachment structure in accordance with another embodiment 130 of the wearable device. This embodiment incorporates a pair of independently rotatable turntables 140a and 140b, with each turntable having an associated electrode mounted thereon. FIG. 8B is a schematic partial cross-sectional view of a portion of the finger ring arrangement shown in FIG. 8A. Unless stated otherwise, the electrodes 134a and 134b and the turntables 140a and 140b may be structured the same as in the embodiment shown in FIGS. 3, 5A, and 5B. FIG. 9 is a flow diagram illustrating a procedure for collecting signal-to-noise (S/N) ratios at a plurality of combinations of rotational orientations of the pair of turntables 140a, 140b. FIGS. 10A-10D are schematic plan views of the embodiment of FIGS. 8A and 8B, illustrating progressive rotation of a second turntable to rotational orientations of 45°, 90°, and 135° relative to the 0° reference rotational orientation shown in FIG. 8A, while the first turntable is held at 0°. FIGS. 10E-10H are schematic plan views of the embodiment of FIGS. 8A and 8B, illustrating progressive rotation of the second turntable to rotational orientations of 0°, 45°, 90°, and 135° relative to the 0° reference rotational orientation after rotation of the first turntable to 45°.

As used herein, a “rotational orientation” of the electrodes 134a and 134b with regard to an embodiment where each of the first and second electrodes 134a and 134b is mounted on an independently rotatable turntable is understood to mean a combination of a rotational orientation of the first electrode 134a and a separate rotational orientation of the second electrode 134b. The first electrode 134a may have a plurality of rotational orientations, with each rotational orientation associated with a respective rotational orientation of a first turntable 140a. Similarly, the second electrode 134b may have a plurality of rotational orientations, with each rotational orientation associated with a respective rotational orientation of a second turntable 140b. Thus, a “rotational orientation” of the turntables 140a, 140b with regard to an embodiment where each of the first and second electrodes is mounted on one of the independently rotatable turntables 140a, 140b is understood to mean a combination of a rotational orientation of the first turntable 140a and a separate rotational orientation of the second turntable 140b. In addition, a “rotational orientation of the at least one turntable” may refer to any of a rotational orientation of a single turntable incorporating both electrodes, and a combination of rotational orientations of both the first and second turntables (in a case where the first and second electrodes are mounted on separate turntables). Also, “rotation of the at least one turntable” may involve rotating any of a single turntable (with both electrodes mounted thereon) and either (or both) of the turntables in an embodiment including two turntables, with each turntable having a single electrode mounted thereon.

FIG. 8A is a schematic plan view of an inner surface of an alternative embodiment 138 of the finger ring attachment structure, incorporating a pair of independently rotatable turntables 140a and 140b, with an electrode 134a mounted on turntable 140a and an electrode 134b mounted on turntable 140b. In FIG. 8A, each turntable is oriented at a reference rotational orientation of 0°. This arrangement may provide a greater number of S/N ratios to choose from in selecting the highest available S/N ratio.

FIGS. 9 and 10A-10H, in combination, illustrate a variation on the S/N ratio acquisition method previously described with regard to FIGS. 5A, 5B, and 6. FIG. 9 is a flow diagram illustrating a procedure for collecting signal-to-noise (S/N) ratios at a plurality of combinations of rotational orientations of turntables 140a and 140b. FIGS. 10A-10D are schematic plan views of the embodiment of FIGS. 8A and 8B, illustrating progressive rotation of a second turntable to rotational orientations of 45°, 90°, and 135° relative to the 0° reference rotational orientation shown in FIG. 8A, while the first turntable is held at 0°. In this scenario, a first turntable 140a is initially fixed in a given rotational orientation while the second turntable 140b is progressively rotated by a predetermined amount equal to 45° between S/N ratio readings as previously described.

Referring to FIG. 9, to begin, a user may start the calibration routine for the wearable device as previously described, by interfacing with an external computing device or by operating a switch (not shown) incorporated into the wearable device.

Next, in block 902, responsive to an instruction generated by the sensor control module 70, the user may rotate both turntables to the same reference rotational orientation (0°, for example) as shown in FIG. 10A. Starting of the calibration routine may also (in block 904) initialize a variable TOTAL1 for turntable 140a by taking the 180/PD1 (where PD1=the predetermined rotation amount for turntable 140a, in degrees) and truncating any fractional portion to obtain an integer value of the quotient. For example, for PD1=35°, 180/PD1=5.14 and TOTAL1=5 (the truncated value of the quotient). For PD1=45° as shown in the example, 180/PD1=4. The variable TOTAL1 becomes the total number of rotational orientations of turntable 140a for which S/N ratio readings should be taken for calibration purposes. Similarly, starting of the calibration routine may initialize a variable TOTAL2 for turntable 140b by taking the quotient 180/PD2 (where PD2=the predetermined rotation amount for turntable 140b, in degrees) and truncating any fractional portion to obtain an integer value of the quotient. For example, for PD2=35°, 180/PD2=5.14 and TOTAL2=5 (the truncated value of the quotient). For PD2=45° as shown in the example, 180/PD2=4. The variable TOTAL2 becomes the total number of rotational orientations of turntable 140b for which S/N ratio readings should be taken for calibration purposes.

Referring to FIG. 9, in a next step (block 906), the sensor control module 70 may determine and store a current rotational orientation of the first turntable 140a. Thus, per the example shown in FIG. 10A, the 0° orientation may be stored as the current orientation of the first turntable 140a. In a next step (block 908), the sensor control module 70 may determine and store a current rotational orientation of the second turntable 140b (initially 0° as shown in FIG. 10A).

In a next step (block 910), the sensor control module 70 may, using an EDA signal generated in the current orientations of the turntables 140a and 140b, determine and store an S/N ratio for the current combination of rotational orientations of the first and second turntables 140a, 140b. This S/N ratio may be stored in a memory 88 in operative association with the current turntable rotational orientations. In this manner, each S/N ratio may be associated with a given combination of rotational orientations of the turntables 140a and 140b. In the example shown, a ratio S/N1 may be stored in association with the combination of 0° (starting) rotational orientations of first and second turntables 140a, 140b shown in FIG. 10A.

Next (block 912), the sensor control module 70 may determine if there is a need to rotate the second turntable 140b to generate an additional sample value of S/N ratio. To this end, the sensor control module 70 may increment a counter CC2 by 1 (indicating that an S/N ratio has just been determined for the current combination of rotational orientations of turntables 140a and 140b) and compare the value of CC2 to the value of TOTAL2. If CC2=TOTAL2, then S/N ratio readings for all the rotational orientations (0°, 45°, 90°, and) 135° of the second turntable 140b have been taken, and no additional S/N ratio readings need to be taken for this portion of the calibration. However, if CC2<TOTAL2, then one or more additional S/N readings should be taken for calibration. The sensor control module 70 may then (in block 914) generate an instruction to rotate the second turntable 140b by the predetermined rotation amount. For the process of acquiring the S/N ratio values, either the first turntable 140a may be fixed first while the second turntable 140b is rotated through the angle values as described, or the second turntable 140b may be fixed first while the first turntable 140a is rotated through the angle values as described. In particular arrangements, the sensor control module 70 may be configured to control a display device (such as a cellular device 111 in operative communication with the sensor control module) or to prompt operation of a display device so as to display the rotation instruction(s) for each of turntables 140a and 140b on the display device. The instruction may include elements such as how to use a turntable rotation tool (not shown) provided with the wearable device 30 and other instructions which may be conveyed clearly in an audio/visual format.

Following generation of the instruction in block 914, the user may proceed to rotate the second turntable 140b responsive to the instruction. The sensor control module 70 may be configured to constantly monitor (in block 916) the rotational orientation of the second turntable 140b (in cooperation with a rotational orientation sensor 37 associated with turntable 140b, for example) to determine when the second turntable 140b has been rotated the predetermined amount (in this case, by 45°). When the second turntable 140b has been rotated the predetermined amount, the sensor control module 70 may (in block 918) generate an instruction to the user to stop rotating the second turntable 140b. Control may then proceed back to block 908 to repeat the steps just described. For example, following the acquisition of an S/N ratio reading at 0°, 0°, second turntable 140b may be rotated 45° to the combination of rotational configurations of the first and second turntables 140a, 140b shown in FIG. 10B. An S/N ratio of S/N2 may be acquired in this combination of rotational configurations. The steps previously described may then be implemented to rotate the second turntable 140b to 90° (FIG. 10C, where an S/N ratio of S/N3 may be acquired), and to 135° (FIG. 10D, where an S/N ratio of S/N4 may be acquired).

At this point, CC2 will have been incremented to 4 (equal to TOTAL2), and the second turntable 140b will be oriented at 135°. Thus, the next time control reaches block 912, there will be no need to further rotate the second turntable 140b. Control may then transfer to block 913, where the sensor control module 70 may generate an instruction to rotate the second turntable 140b back to the reference orientation 0°, and to increment the counter CC1 for the first turntable 140a (indicating that all S/N ratio values for a current rotational orientation (in this case, 0°) of the first turntable have been acquired). Control may then proceed to block 920, where the sensor control module 70 may determine if there is a need to rotate the first turntable 140a to generate additional sample value(s) of S/N ratio. When there is no need to rotate the first turntable 140a to generate additional sample value(s) of S/N ratio (i.e., when CC1=TOTAL1=4), the sensor control module 70 may (in block 926) generate message indicating all of the needed S/N ratio values have been obtained (i.e., that data collection is complete). However, in the present case, since CC1 is less than TOTAL1, the sensor control module 70 may determine that there is a need to rotate the first turntable 140a to generate one or more additional sample value(s) of S/N ratio. Control may then proceed to block 922, where the sensor control module 70 may generate an instruction to rotate the first turntable 140a by the predetermined rotation amount. Execution of blocks 913, 920, and 922 provides the combination of rotational orientations shown in FIG. 10E.

Following generation of the instruction in block 922, the user may proceed to rotate the first turntable 140a responsive to the instruction. The sensor control module 70 may be configured to constantly monitor (in block 924) the rotational orientation of the first turntable 140a (in cooperation with a rotational orientation sensor 37 associated with turntable 140a, for example) to determine when the first turntable 140a has been rotated the predetermined amount (in this case, 45°). When the first turntable 140a has been rotated the predetermined amount, the sensor control module 70 may (in block 928) generate an instruction to stop rotating the first turntable 140a. Control may then proceed back to block 906, where the sensor control module 70 may determine and store a current rotational orientation of the first turntable 140a (which is now in the 45° orientation as shown in FIG. 10E). Thus, per the example shown in FIG. 10E, the 45° orientation may be stored as the current orientation of the first turntable 140a.

Next, control may transfer again to block 908, where the sensor control module 70 may determine and store a current rotational orientation of the second turntable 140b (initially 0° as shown in FIG. 10E). The sensor control module 70 may then determine an S/N ratio S/N5 at 45°, 0°. With the first electrode now oriented at 45°, blocks 910 through 918 may be executed as previously described to determine an S/N ratio S/N6 at a combined rotational orientation of 45°. 45° (FIG. 10F), an S/N ratio S/N7 at a combined rotational orientation of 45°, 90° (FIG. 10G), and an S/N ratio S/N8 at a combined rotational orientation of 45°, 135° (FIG. 10H). When the S/N ratio S/N& has been acquired, the counter CC2 will again have incremented to where CC2=TOTAL2. At this point, a need will be determined (in block 920) to rotate the first turntable 140a to the next rotational orientation (i.e., 90°). The first turntable 140a may then be rotated to 90° and the second turntable back to 0°, after which acquisition of additional S/N ratios will proceed.

The process just described may be repeated until four S/N ratios for each of rotational orientations 0°, 45°, 90°, and 135° of the first turntable 140a have been acquired by successively rotating the second turntable 140b to rotational orientations of 0°, 45°, 90°, and 135° as described, thereby generating a total of sixteen S/N ratios, with each S/N ratio associated with a unique combined orientation of the turntables. Determination of the highest S/N ratio may then be determined as previously described with respect to FIG. 7.

Thus, in the wearable device embodiment just described, the wearable device includes first and second independently rotatable turntables, and a single electrode affixed to each turntable. The embodiment may also include a processor(s) and a memory communicably coupled to the processor(s). The memory may store a sensor control module including computer-readable instructions that when executed by the processor(s) cause the processor(s) to, using an EDA signal generated while the first and second turntables are in respective first and second current rotational orientations, determine an S/N ratio associated with a combination of the of first and second current rotational orientations. The sensor control module may also be configured to determine if a need exists to rotate one or more of the turntables a predetermined amount from the current rotational orientation of the turntable(s). The sensor control module may also be configured to, if a need exists to rotate one or more turntable(s) a predetermined amount from the current rotational orientation of the turntable(s), generate an instruction(s) to rotate the one or more turntable(s) the predetermined amount.

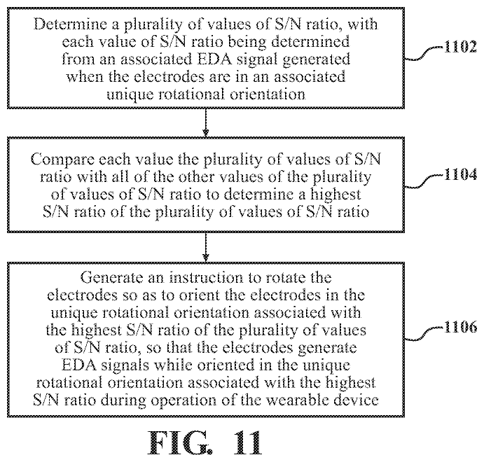

FIG. 11 is a flow diagram illustrating a method of determining electrodermal activity (EDA) associated with a user of a wearable device (such as device 30) incorporating a pair of electrodes (such as electrodes 34a, 34b) rotatably mounted to an attachment structure of the wearable device. In one or more arrangements, the sensor control module 70 and the processor(s) 80 may be configured to perform the method described in FIG. 11. Referring to FIG. 11, in a first step (block 1102), a plurality of values of S/N ratio may be determined in a manner previously described, with each value of S/N ratio being determined from an associated EDA signal generated when the electrodes are in an associated unique rotational orientation. Next (in block 1104), each value the plurality of values of S/N ratio may be compared with all of the other values of the plurality of values of S/N ratio to determine a highest S/N ratio of the plurality of values of S/N ratio. In a next step (block 1106), an instruction may be generated to rotate the electrodes so as to orient the electrodes in the rotational orientation associated with the highest S/N ratio of the plurality of values of S/N ratio, so that the electrodes generate EDA signals while oriented in the rotational orientation associated with the highest S/N ratio during operation of the wearable device.

In yet another aspect of the embodiments described herein, a non-transitory computer-readable medium is provided for determining electrodermal activity (EDA) associated with a user of a wearable device incorporating a pair of electrodes rotatably mounted to an attachment structure of the wearable device. The non-transitory computer-readable medium stores instructions that when executed by a processor(s) cause the processor(s) to determine a plurality of values of S/N ratio, with each value of S/N ratio being determined from an associated EDA signal generated when the electrodes are in an associated unique rotational orientation. Further instructions cause the processor(s) to compare each value the plurality of values of S/N ratio with all of the other values of the plurality of values of S/N ratio to determine a highest S/N ratio of the plurality of values of S/N ratio. Further instructions cause the processor(s) to generate an instruction to rotate the electrodes so as to orient the electrodes in the rotational orientation associated with the highest S/N ratio of the plurality of values of S/N ratio, so that the electrodes generate in EDA signals while oriented in the rotational orientation associated with the highest S/N ratio during operation of the wearable device.

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-8, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises all the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain, or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor(s), a RAM, a ROM, a flash memory, or another suitable electronic storage medium. In still further aspects, a module, as envisioned by the present disclosure, is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, RF, etc., or any suitable combination of the foregoing. Computer program code for carrying out operations for aspects of the present arrangements may be written in any combination of one or more programming languages, including an object-oriented programming language such as Java™, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . and . . . .” as used herein refers to and encompasses any and all possible combinations of one or more of the associated listed items. As an example, the phrase “at least one of A, B, and C” includes A only, B only, C only, or any combination thereof (e.g., AB, AC, BC or ABC).

Aspects herein can be embodied in other forms without departing from the spirit or essential attributes thereof. Accordingly, reference should be made to the following claims, rather than to the foregoing specification, as indicating the scope hereof.

What is claimed is:

1. A wearable device comprising:
an attachment structure; and
at least one turntable mounted to the attachment structure; and
a pair of insulatively spaced-apart electrodes affixed to the at least one turntable and structured to contact a skin surface of a user.

2. The wearable device of claim 1, wherein the attachment structure is a finger ring.

3. The wearable device of claim 1, wherein a surface of a first electrode is structured to be coplanar with a surface of a second electrode.

4. The wearable device of claim 3, wherein a plane defined by a surface of an insulator separating the first electrode from the second electrode is spaced apart from a plane including the surface of the first electrode and the surface of the second electrode.

5. The wearable device of claim 1, wherein the pair of electrodes is affixed to a single turntable, and wherein the wearable device further comprises:
a processor(s); and
a memory communicably coupled to the processor(s) and storing a sensor control module including computer-readable instructions that when executed by the processor(s) cause the processor(s) to:
using an electrodermal activity (EDA) signal generated while the turntable is in a current rotational orientation, determine an S/N ratio associated with the current rotational orientation;
determine if a need exists to rotate the turntable a predetermined amount from the current rotational orientation; and
if a need exists to rotate the turntable a predetermined amount from the current rotational orientation, generate an instruction to rotate the turntable the predetermined amount.

6. The wearable device of claim 5, wherein the sensor control module further includes computer-readable instructions that when executed by the processor(s) cause the processor(s) to control (or prompt operation of) a display device so as to display the instruction to rotate on a display device.

7. The wearable device of claim 5 wherein the sensor control module further includes computer-readable instructions that when executed by the processor(s) cause the processor(s) to:
determine when the turntable has been rotated the predetermined amount; and
responsive to a determination that the turntable has been rotated the predetermined amount, generate an instruction to stop rotating the turntable.

8. The wearable device of claim 1, further comprising:
a processor(s); and
a memory communicably coupled to the processor(s) and storing a sensor control module including computer-readable instructions that when executed by the processor(s) cause the processor(s) to:
determine a plurality of values of S/N ratio, each value of S/N ratio being associated with a different rotational orientation of the at least one turntable;
compare each value the plurality of values of S/N ratio with all of the other values of the plurality of values of S/N ratio to determine a highest S/N ratio of the plurality of values of S/N ratio; and
generate an instruction to rotate the at least one turntable so as to orient the at least one turntable in a rotational orientation associated with the highest S/N ratio of the plurality of values of S/N ratio.

9. The wearable device of claim 8, wherein the sensor control module further includes computer-readable instructions that when executed by the processor(s) cause the processor(s) to control (or prompt operation of) a display device so as to display the instruction to rotate on a display device.

10. The wearable device of claim 1, further comprising first and second independently rotatable turntables, and a single electrode affixed to each turntable.

11. The wearable device of claim 10, further comprising:
a processor(s); and
a memory communicably coupled to the processor(s) and storing a sensor control module including computer-readable instructions that when executed by the processor(s) cause the processor(s) to:
using an EDA signal generated while the first and second turntables are in respective first and second current rotational orientations, determine an S/N ratio associated with a combination of the of first and second current rotational orientations;
determine if a need exists to rotate one or more of the turntables a predetermined amount from the current rotational orientation of the turntable(s); and
if a need exists to rotate one or more turntable(s) a predetermined amount from the current rotational orientation of the turntable(s), generate an instruction(s) to rotate the one or more turntable(s) the predetermined amount.

12. An electrodermal activity (EDA) sensor, comprising:
at least one turntable; and
a pair of insulatively spaced-apart electrodes affixed to the at least one turntable and structured to contact a skin surface of a user.

13. A method of determining electrodermal activity (EDA) associated with a user of a wearable device incorporating a pair of electrodes rotatably mounted to an attachment structure of the wearable device, the method comprising steps of:
determining a plurality of values of S/N ratio, with each value of S/N ratio being determined from an associated EDA signal generated when the electrodes are in an associated rotational orientation;
comparing each value the plurality of values of S/N ratio with all of the other values of the plurality of values of S/N ratio to determine a highest S/N ratio of the plurality of values of S/N ratio; and
generating an instruction to rotate the electrodes so as to orient the electrodes in the rotational orientation associated with the highest S/N ratio of the plurality of values of S/N ratio, so that the electrodes generate EDA signals while oriented in the rotational orientation associated with the highest S/N ratio during operation of the wearable device.
